(19) United States
(12) Patent Application Publication
(10) Pub. No.: US 2026/0298816 A1
(43) Pub. Date: Oct. 1, 2026

(54) METHOD AND SYSTEM FOR DETERMINING THE CONCENTRATION OF ONE OR MORE SUGARS IN TOBACCO LEAVE SAMPLES

(71) Applicant: British American Tobacco (Investments) Limited, London (GB)

(72) Inventors: Marcelo Caetano Alexandre MARCELO, São Leopoldo (BR); Maurilio Gustavo NESPECA, Porto Alegre (BR); Samuel KAISER, Porto Alegre (BR)

(21) Appl. No.: 19/477,051

(22) PCT Filed: Apr. 19, 2024
(86) PCT No.: PCT/EP2024/060804

§ 371 Date: Oct. 20, 2025

(30) Foreign Application Priority Data
Apr. 20, 2023 (BR) 1020230076157
Apr. 20, 2023 (GB) 2305843.1

(51) Int. Cl. G01N 21/3563; G01N 21/84; G01N 33/00

(52) U.S. Cl. CPC G01N 21/3563; G01N 21/84; G01N 33/0098; G01N 2021/8466

(57) ABSTRACT

A computer-implemented method for detecting a concentration of one or more sugars in tobacco target samples using a target analysis technique based on near-infrared spectroscopy is provided, the method comprising providing one or more trained artificial intelligence models obtained or obtainable by training one or more untrained or pre-trained artificial intelligence models using sample information, reference concentration data relating to the one or more sugars, and near-infrared reference spectral data obtained or obtainable using the target analysis technique for a plurality of tobacco reference samples; analyzing the tobacco target samples using the target analysis technique providing near-infrared target spectra and preprocessing the near-infrared target spectra to obtain preprocessed near-infrared target spectral data; and deriving the concentration of the one or more sugars in the tobacco target samples using the or one of the trained artificial intelligence models and on the basis of the preprocessed near-infrared target spectral data; wherein said analyzing the tobacco target samples includes providing a plurality of the near-infrared target spectra in a common sub-range of the near-infrared wavelength range for each of the tobacco reference samples, wherein said preprocessing the near-infrared target spectra includes averaging said plurality of near-infrared target spectra for each of the tobacco target samples and performing a standard normal variates transformation followed by Savitzky-Golay first derivative and mean centering, and wherein said tobacco target samples include one or more groups of tobacco target samples grouped according to chemical similarity of different tobacco varieties or types, at least one trained artificial intelligence model being provided for each of the groups or tobacco varieties or types therein. A method of providing one or more trained artificial intelligence models, a system and a computer program are provided as well.

### CROSS-REFERENCE TO RELATED APPLICATIONS

This present application is a National Phase entry of PCT Application No. PCT/EP2024/060804, filed Apr. 19, 2024, which claims priority from Brazilian Application No. 1020230076157, filed Apr. 20, 2023, and which claims priority from Great Britain Application No. 2305843.1, filed Apr. 20, 2023, each of which are fully incorporated herein by reference in their entireties.

### TECHNICAL FIELD

The present invention relates to a method for analyzing tobacco leave samples, to details of implementing such a method, particularly including the use of artificial intelligence, and to a corresponding analysis system.

### BACKGROUND

Sugars are important compounds in natural products, where they primarily serve as a source of energy, but also play an important role as precursors of flavours or bioactive compounds. The sugar content of tobacco depends on the tobacco variety, the crop, and particularly on the curing conditions (including temperature, duration, and humidity). If high temperatures are used during curing of tobacco leaves (such as in flue and sun curing), the final sugar content is typically high. Conversely, air drying at lower temperatures results in a generally lower final sugar content of the tobacco product.

In addition to sugars in the strict sense, i.e., simple sugars, di- and oligosaccharides, polysaccharides such as cellulose, starch, and pectin are also detected in tobacco, and glucose may be used as a reference compound. Degradation of polysaccharides results in a higher yield of simple sugars, but at the same time reduces the oxidation of sugars and their conversion to carbon dioxide and water. The loss of sugar may be compensated by added sugars to mask undesirable taste characteristics and to achieve a better and more pleasant taste during smoking. However, the carbohydrates in tobacco can be precursors to undesired compounds, including formaldehyde and 5-hydroxymethylfurfural. Therefore, the content and composition of carbohydrates is relevant to the product properties and quality in connection with the formation of undesired compounds.

Analysis of total sugars in tobacco may, according to Recommended Method No. 89 of the Cooperation Centre for Scientific Research Relative to Tobacco (CORESTA), April 2019, Ref. RAC-054-2-CRM-89, be performed using a continuous flow analysis method using hydrochloric acid (HCl) for hydrolysis and p-hydroxybenzoic acid hydrazide (PAHBAH) for colour formation. This method is applicable to unprocessed tobacco lamina and processed tobacco such as cigarette blend tobacco and roll-your-own (RYO) tobacco.

### SUMMARY

Embodiments as disclosed herein relate to a computer-implemented method for detecting a concentration of one or more sugars in tobacco target samples using a target analysis technique based on near-infrared spectroscopy. In such embodiments, one or more trained artificial intelligence models obtained or obtainable by training one or more untrained or pre-trained artificial intelligence models using sample information, reference concentration data relating to the one or more sugars, and near-infrared reference spectral data obtained or obtainable using the target analysis technique for a plurality of tobacco reference samples may be provided. The tobacco target samples may be analyzed using the target analysis technique providing near-infrared target spectra and preprocessing the near-infrared target spectra to obtain preprocessed near-infrared target spectral data. The concentration of the one or more sugars in the tobacco target samples may be derived using the, or one of the, trained artificial intelligence models and based on the preprocessed near-infrared target spectral data. According to some embodiments, the tobacco target samples and said analyzing the tobacco target samples includes providing a plurality of the near-infrared target spectra in a common sub-range of the near-infrared wavelength range for each of the tobacco reference samples.

Said preprocessing the near-infrared target spectra may include averaging said plurality of near-infrared target spectra for each of the tobacco target samples and at least one of performing a standard normal variate transformation, forming a first or second order derivative, performing a multiplicative scatter correction, performing an orthogonal signal correction, performing a baseline correction, smoothing, and applying a multivariate filter. Said tobacco target samples could generally include one or more groups of tobacco target samples, which is, however, particularly no mandatory feature of the present invention. Such groups could be the result of a grouping of tobacco varieties or tobacco types according to chemical similarities. These could, for example, tobacco samples which are cured according to specific curing conditions, i.e. one group could include tobacco varieties or tobacco types that are typically flue-cured (such as Virginia and Amarelinho tobacco varieties) and another group could include those that are typically air-cured (such as Burley, Dark, Comum and Maryland varieties). For each group, the members of the group, i.e. the tobacco varieties or tobacco types, could be chemically more similar than that of a different group. For each group, or for each tobacco variety or tobacco type within the groups, at least one trained artificial intelligence model could be provided. While such grouping was found to be advantageous for nicotine and total alkaloid measurements, which is not an aspect of the present invention, analysing total sugars, such as proposed herein, was not found to provide significant advantages for total sugar analysis. In embodiments of the present invention, a standard normal variate transformation, followed by forming a Savitzky-Golay first derivative and mean centering were found to be particularly advantageous for preprocessing.

According to some embodiments, the one or more sugars are selected from total sugars present in the tobacco target samples, and glucose. The plurality of target spectra may be averaged for each of the tobacco target samples may be three according to some embodiments. Said preprocessing, according to some embodiments, may further comprise forming a first derivative, wherein a window size of fifteen and an order of two may particularly be used in forming the first derivative, according to some embodiments. In some embodiments, the common sub-range of the near-infrared wavelength range referred to above may be between 947 nm and 1,618 nm.

Said analyzing the tobacco target samples may, according to some embodiments, particularly include spectroscopically examining tobacco leaves at a plurality of predetermined sampling positions. Furthermore, according to some embodiments, analyzing the tobacco target samples may be performed under defined moisture conditions. The tobacco target samples may comprise one or more green tobacco leaves which may, according to some embodiments, be probed under defined probing conditions. These may be leaves which may later be harvested. In embodiments of the invention, only samples used for building, calibrating or validating the models need to be collected or harvested, because the reference results are required. Except these situations, green leaves can be analyzed by the calibrated near infrared models without harvesting them.

In certain embodiments, the method may include a partial least squares regression in connection with the artificial intelligence model or models. Generally, partial least squares-discriminant analysis also can be used for a qualitive analysis in this application. For example, partial least squares-discriminant analysis can be applied to discriminate samples with low level of sugars and samples with high level of sugars, for example. The method may, in some embodiments, be performed in a system comprising a near-infrared spectroscopy device and a mobile unit connected or connectable to a server, wherein the trained artificial intelligence model is downloaded from the server to the mobile unit to perform at least said deriving the concentration of the one or more sugars in the tobacco target samples.

A method of providing one or more trained artificial intelligence models usable in a method of which some embodiments are explained herein is also proposed. The method comprises providing a plurality of tobacco reference samples and sample information indicating characteristics of the tobacco reference samples, analyzing the tobacco reference samples for the one or more sugars using one or more reference analysis techniques providing reference concentration data, analyzing each of the tobacco reference samples using the target analysis technique providing near-infrared reference spectra, preprocessing the near-infrared reference spectra for the tobacco reference samples obtaining preprocessed spectral data, and training one or more artificial intelligence models using the sample information, the reference concentration data and the preprocessed spectral data obtaining the trained artificial intelligence model. In some embodiments, said analyzing the tobacco reference samples and said preprocessing the near-infrared reference spectra may be performed as for the tobacco target samples. As mentioned, and in contrast to methods for analysing alkaloids, for example, the tobacco reference samples not mandatorily need to include different groups of tobacco target samples grouped according to chemical similarities of tobacco varieties or tobacco types, and one trained artificial intelligence model may be provided for all tobacco samples of different groups when analysing sugars, as proposed herein in embodiments.

A trained artificial intelligence model stored on a non-tangible computer-readable medium obtained or obtainable by using a method according to claim may also be provided.

Embodiments disclosed herein may also relate to a trained artificial intelligence model of which some embodiments are explained herein, which is particularly a partial least square regression algorithm.

A system for detecting a concentration of one or more sugars in tobacco target samples using a target analysis technique based on near-infrared spectroscopy is proposed as well according to some embodiments, the system being configured to perform a method of which some embodiments are explained herein.

Such system may, in some embodiments, comprise a near-infrared spectroscopy device and a mobile unit connected or connectable to a server, wherein the trained artificial intelligence model may be downloadable from the server to the mobile unit and wherein the mobile unit may be configured to perform at least said deriving the concentration of the one or more sugars in the tobacco target samples.

Further embodiments relate to a computer program with a program code for performing a method of which embodiments are explained herein when the computer program is run on a processor. A tangible, non-transitory computer-readable medium having instructions thereon, which, upon execution by one or more hardware processors, facilitates execution of the steps of a corresponding method may also be provided.

### BRIEF DESCRIPTION OF THE FIGURES

Embodiments of the invention will now be described, by way of example only, with reference to accompanying drawings, in which:

FIG. 1 illustrates a system usable according to some embodiments.

FIG. 2 illustrates a method development process according to some embodiments.

FIG. 3 illustrates a correlation plot according to some embodiments.

FIG. 4 illustrates an analysis according to some embodiments.

FIG. 5 illustrates providing an artificial intelligence model according to some embodiments.

### EMBODIMENTS OF THE INVENTION

Obtaining chemical information on green tobacco may require, according to conventional methods, several steps, such as collection of representative leaves, refrigerated storage, transportation, drying, stalk removal, milling and analysis by laboratory reference methods, usually based on chemical reactions followed by spectroscopic, infrared or chromatographic analyses. In addition to the long time, handling, and cost involved in conventional methods, the acquisition of chemical information on samples in places far from the laboratories is very difficult. Thus, the use of near-infrared spectroscopic analysis, particularly using portable devices, brings the benefit of analysis in remote locations, more representative sampling and real-time information acquisition.

Using embodiments of the method proposed herein, which may be computer-implemented and may be suitable for determining a concentration of one or more sugars in tobacco target samples using a target analysis technique based on near-infrared spectroscopy, these disadvantages may be overcome. Such embodiments may include performing an analysis for one or more sugars, such as glucose, directly on the tobacco field, for which reasons the sample preparation and analysis steps as explained above can be dispensed of. This reduces manual error, storage, and processing errors. Methods according to embodiments of the present invention therefore allow for a more robust, easy, and reliable analysis of sugars and therefore an early assessment of sugar contents of tobacco. Due to the ease of analysis including near-infrared spectroscopy, sugar contents may be analyzed with a substantially higher frequency, as essentially no transportation, sample preparation and processing effort is present. This allows for a tight and fine-grained monitoring of the sugar content of tobacco which, as mentioned, may vary over the growth period of a tobacco plant and may be decisive for the features of the final tobacco product. Embodiments particularly relate to analyzing tobacco leaves in the field.

If, herein, reference is made to “green” tobacco leaves, this term shall particularly denote tobacco leaves which not yet harvested from the plant, or which are harvested not more than one, two, three or five hours before, such that no deterioration or substantial change in the composition of components has occurred. According embodiments of the present invention, the model used includes tobacco leaves that are 50 to 135 days old (counting from the date the tobacco plant was transplanted into the farm soil). However, the scope of the model can still be expanded to more or less days after transplanting.

In methods according to some embodiments as mentioned, one or more trained artificial intelligence models obtained or obtainable by training one or more untrained or pre-trained artificial intelligence models using sample information, reference concentration data relating to the one or more sugars, and near-infrared reference spectral data obtained or obtainable using the target analysis technique for a plurality of tobacco reference samples may be provided, as mentioned. A “pre-trained” artificial intelligence model is particularly adapted to training according to some embodiments, in any manner conceivable. Further details as to artificial intelligence models which may be used according to some embodiments are given above and further explained below. Such embodiments are generally not limited to a specific type of artificial intelligence models, even if certain embodiments as disclosed and discussed herein are used in connection with specific types. As the artificial intelligence models, commercially or freely available, “standard” artificial intelligence models or artificial intelligence frameworks may be used, but other embodiments may also be used in connection with specifically designed and/or adapted models.

Models used according to embodiments of the present invention particularly may include a partial least square regression algorithm (PLS). This algorithm may particularly advantageously used for the development of prediction models with near-infrared data. The main reason is its ability to handle the high covariance of spectral data by compressing the spectral variables into latent variables, which explain the variance into spectral data and property of interest. In addition, the PLS model has other advantages such as low computational cost, simplicity in predicting new samples, easy interpretation, verification of spectral residuals, association with variable selection algorithms, among others.

Although PLS is a particularly advantageous option, other algorithms can be used to correlate spectra with chemical concentration such as: support vector machine (SVM), artificial neural network (ANN), decision trees, principal component regression (PCR), locally weighted regression (LWR), and multiple linear regression (MLR).

In some embodiments, the tobacco target samples may be analyzed using the target analysis technique providing near-infrared target spectra, i.e., essentially the same method, which is later used for analyzing the target samples, and preprocessing the near-infrared target spectra to obtain preprocessed near-infrared target spectral data. Embodiments relate to specific preprocessing steps which have shown to have several specific advantages, and which allow for a particularly well correlation of near-infrared spectra with an sugar content determined by reference measurements. The concentration of the one or more sugars in the tobacco target samples may therefore be advantageously derived using the or one of the trained artificial intelligence models and based on the preprocessed near-infrared target spectral data according to some embodiments.

According to some embodiments, said analyzing the tobacco target samples includes providing a plurality of the near-infrared target spectra in a common sub-range of the near-infrared wavelength range for each of the tobacco reference samples. This has proven to significantly improve correlations and therefore the reliability and preciseness of the method Said preprocessing the near-infrared target spectra may, as was found particularly advantageous in some embodiments, averaging said plurality of near-infrared target spectra for each of the tobacco target samples and performing a standard normal variates transformation. According to some embodiments, a further improvement could be achieved when one or more groups of tobacco target samples were grouped according to chemical similarities in tobacco varieties or tobacco types, and when at least one trained artificial intelligence model was provided for each of the groups or tobacco varieties or types therein.

Some embodiments provide a portable handheld solution to assess chemical composition of particularly green tobacco which, however, can also be employed for cured tobacco and tobacco-related products. The solution particularly may comprise a portable near-infrared device and, in some embodiments, a cellphone and a machine learning application.

In some embodiments, an analytical platform based on a real-time chemical methodology including machine learning algorithms is provided. By using an infrared portable device, a cellphone application, and a machine learning solution, an improved assessment of the chemical composition of tobacco and tobacco related products is possible according to some embodiments. The analytical methodology may involve, in some embodiments, a) a superficial chemical analysis of a tobacco sample (leaves, cut rag, pouches, recon, cigarettes, etc.) through an portable infrared spectroscopy device; b) a cellphone application that connects through a suitable short-range radio communication technique with the spectroscopy device and transforms the output through signal preprocessing techniques and machine learning algorithms in a measurement of tobacco chemical composition (alkaloids, sugars, amino acids, chlorophyll, glycerol, and others); and c) providing reports and data management tools bases on these chemical contents. The chemical methodology may provide measurement results on the cellphone screen and store in a database for further comparisons. It can be implemented in any process step of tobacco production, in the field, cigarette manufactory or even be used at commercial products, if calibrated to do so.

Before further turning to specific embodiments, some general background on methodologies and devices that may be used in such embodiments will be provided. Be it noted that all technical details and variants thereof as explained hereinbelow may form part of specific embodiments, but the embodiments are not limited to by these explanations and may alternatively or additionally include other technical details.

Near-Infrared Spectroscopy, NIR spectroscopy or NIRS for short, which is used in some embodiments, is a physical analysis technique based on spectroscopy in the short-wave infrared light range. Near-infrared spectroscopy may essentially resemble infrared spectroscopy used in the Mid-Infrared (MIR) and Far-Infrared (FIR) range but allows the use of other materials and radiation sources. However, near-infrared spectroscopy usually offers, in some embodiments, easier access and other forms of analysis.

Like other vibrational spectroscopy methods, near-infrared spectroscopy is based on the excitation of molecular vibrations by electromagnetic radiation. In near-infrared spectroscopy, detection takes place in the near infrared (760 to 2,500 nm) region of the electromagnetic spectrum. In this wavelength range, overtone, or combination vibrations of the basic molecular vibration from the mid-infrared may be observed. These are, in some embodiments, not interpreted directly when analyzing samples but are evaluated with the help of statistical and/or artificial intelligence methods. For quantitative determinations, data sets with known content or known concentrations of the substance of interest are created beforehand in some embodiments of infrared spectroscopy.

Near-infrared spectroscopy devices may, in certain embodiments, be provided in a portable form, the term “portable” particularly relating to an autonomous or rechargeable energy source such as rechargeable batteries, and an overall configuration which allows for holding the device with one or two hands. A portable device may particularly include user-interface means in an integrated form, i.e., a portable device particularly does not include or is not, or at least not permanently, connected to an external device such as a screen, a keyboard, a mouse, etc., as further explained below.

Near-infrared spectroscopy may be used for a variety of purposes such as for determining the water content in all kinds of products. Near-infrared spectroscopy may further be used in quality analyses of agricultural products (grain, flour, milk, oil fruits) and animal feed for the determination of moisture, proteins, crude fibers, and fat content, but also for purposes such as determining to carboxy groups in plastics. A further field of application is process control in the food industry, but also in processes for producing chemical and pharmaceutical products and petrochemistry. In the chemical industry, near-infrared spectroscopy is widely used in process control, for example, for online analysis of intermediate and final products, especially in esterification reactions. Another application is waste separation, where items such as beverage cartons, composites and the various types of plastics may be detected and sorted out of the waste stream.

Near-infrared spectroscopy typically is performed as diffuse reflectance spectroscopy to obtain molecular spectroscopic information. In reflectance spectroscopy techniques, a reflectance spectrum may be obtained by the collection and analysis of reflected electromagnetic radiation as a function of frequency. Generally, two different types of reflection on a surface may be observed, which are regular or specular reflection which is usually associated with reflection from smooth, polished surfaces like mirrors, and diffuse reflection associated with reflection from so-called mat or dull surfaces textured like powders or biological surfaces such as the epidermis of plant leaves. Techniques such as external reflectance and total internal reflectance spectroscopy use the phenomenon of specular reflection to obtain spectroscopic information. In diffuse reflectance spectroscopy, in contrast, electromagnetic radiation reflected from dull surfaces is collected and analyzed. If a sample to be analyzed is not shiny, and is not amenable to conventional transmission spectroscopy, diffuse reflectance spectroscopy may be an advantageous alternative. Some embodiments particularly include diffuse reflectance spectroscopy.

In certain embodiments, a portable near-infrared spectroscopy device may be designed for solids analysis by (diffuse) reflectance spectroscopy. Since the solid sample consists of organic matter, i.e., molecules that have bonds between carbon and hydrogen atoms and may contain some heteroatoms (C—C, C—H, C—O, C═O, etc.), the sample will present responses in the infrared range. Such a portable near-infrared spectroscopy device may work in the Short-Wave Infrared Region (SWIR), between 900 nm to 1,700 nm wavelength. Although this spectral region has low sensitivity compared to wider wavelength ranges (1,700 to 2,500 nm), there are reflectance signals from combination bands of different chemical groups, in addition to 3rd and 4th overtones (harmonic signals), as mentioned. Therefore, several chemical compounds and properties of organic samples can be determined through chemometric models in this spectral range in certain embodiments.

Organic compounds do not present a single (univariate) signal in near infrared spectroscopy, but rather a combination of signals that may or may not covariate with the sample matrix. Thus, the determination of a chemical compound by near-infrared spectroscopy may be performed, in some embodiments, using multivariate models that correlate the spectral signals with the sample composition. Multivariate models can be developed to quantify compounds (regression models) or to classify samples (pattern recognition models). Several regression and classification methods can be used for the development of models, such as regression or classification by Partial Least Squares (PLS), Support Vector Machine (SVM), Artificial Neural Networks (ANN), classification by Soft Independent Modeling by Class Analogy (SIMCA), among others. Regardless of the multivariate method, the development of the model in certain embodiments may include two matrices that make a database: (1) near-infrared spectra; and (2) properties of interest of the respective spectra.

In embodiments, a model is developed which is robust enough to internal and external interferences. For this, the database is made to cover as much variance as possible. For example, database samples are, in certain embodiments, be as representative as possible; the property of interest, in certain embodiments, covers the maximum and minimum expected values; and, in certain embodiments, the spectra acquisition conditions are made to be coherent with the conditions expected in the application (different devices, analysts, environmental conditions, etc.). Since a model obtained accordingly is robust and well validated, it can be applied to new samples.

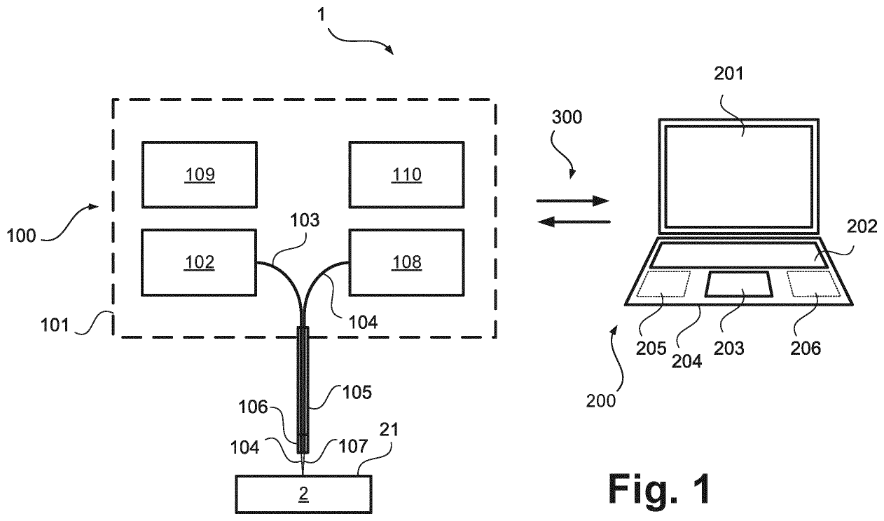

In FIG. 1, a system 1 usable according to some embodiments is schematically illustrated. The system 1 comprises, in the example shown, and in some embodiments, a near-infrared spectroscopy device 100, which is shown is being used to analyze a sample 2, such as a tobacco leaf, and a computer system 200 which is shown to be connected with the spectroscopy device 100 via one or more wired or wireless connections 300 which may or may not be part of system 1 and which may be provided in any manner conceivable, e.g., using solid cables, short, intermediate or long-distance radio technology and/or connections via local, regional or global networks, such as the internet (not specifically illustrated). Certain embodiments include, as mentioned, using a portable near-infrared spectroscopy device.

While computer system 200 is illustrated for reasons of clarity as a laptop computer with typical components such as a screen 201, a keyboard 202, a trackpad 203, a housing 204, a processor 205 and a storage unit 206 such as a hard disk integrated in the housing 204, it, or any part thereof, may in some embodiments be part of the near-infrared spectroscopy device 100, i.e., be provided in a common constructional unit such as a housing 101 of the near-infrared infrared spectroscopy device 100. That is, the computer system 200 may be part of or coupled to the near-infrared spectroscopy device 100 in any manner conceivable and the illustration in FIG. 1 is not intended to be limiting the scope of the present invention. Particularly, the near-infrared spectroscopy device 100 may be provided, in some embodiments, as a portable near-infrared spectroscopy device 100 in which at least a part of the user interface means found in a computer system 200 may be provided in a suitable form, such as a touchscreen, a keypad, etc. Alternatively, at least parts of computer system 200 may be provided by a portable device such as a smartphone, a tablet computer, etc., or components may be distributed among different units.

The near-infrared spectroscopy device 100 comprises, in some embodiments, and integrated into the housing 101, an illumination unit 102 which may be configured to provide illumination light 104 in a wavelength suitable for near-infrared spectroscopy. Illumination unit 102 may comprise any light source, such as a light emitting diode, and an any means or combination of means adapted for spatial or temporal light modulation, which are not shown for reasons of conciseness, and may be configured to couple illumination light into an illumination light guide 103 such as an optical fiber. Illumination light guide 103 may be arranged, together with or separately from, a detection light guide 104, in a sheave 105, which may be arranged in comprising a terminal examination head 106 which may be directed and/or oriented, in some embodiments, to, or in relation to, a sample 2 such that illumination light 104 may be irradiated onto a surface 21 of the sample 2.

Light 107, particularly diffusely reflected light, from the surface 21 of the sample 2 may be coupled, using the detection light guide 104, into a detection unit 108. In some embodiments, detection unit 108 may comprise any means or combination of means adapted for spatial or temporal light modulation, such as, but not limited to, lenses, filters, mirrors, prisms, etc., and may comprise a spectrally resolving detector as suitable for acquiring near-infrared spectra. Spectra or raw spectra acquired by detection unit 108 may, within the near-infrared spectroscopy device 100 using a processing unit 109, or external thereof, e.g., in computer system 200 or in a cloud computing unit, be at least one of analyzed, processed, displayed, and stored, such as further explained below for some embodiments, e.g., using aspects of artificial intelligence. In some embodiments, processing unit 109 may also be adapted or configured for communication with an external device, such as computer system 200, the internet, a mobile telephone network or any other means of communication.

Other embodiments of a near-infrared spectroscopy device 100 may lack an illumination and/or detection light guide 103, 104 and a corresponding sheath but may adapted to irradiate illumination light and receive reflected light through a transparent window of the housing 101 of the near-infrared spectroscopy device 100, making an even more compact design possible. There is no limitation as to the specific configuration of the near-infrared spectroscopy device 100 in embodiments discussed herein.

The detection unit 108 may, in other words, and in some embodiments, be configured to acquire near-infrared spectra and may be coupled to the computer system 200 or may be equipped with a processing unit 109. For reasons of conciseness, reference will now be made to the computer system 200 but similar or essentially the same explanations may apply to a processing unit 109 or a processing system distributed between the near-infrared spectroscopy device 100 and the computer system 200 and/or over a network.

The computer system 200 may be configured to perform at least a part of a method according to some embodiments described herein. The computer system 200 may be configured to execute a machine learning algorithm. The computer system 200 and the near-infrared spectroscopy device 100 may be separate devices but may also be combined in a common package or housing 101, as mentioned. The computer system 200 may be part of a central computing system of the near-infrared spectroscopy device 100. Additionally, or alternatively, the computer system 200 may be part of a sub-component of the near-infrared spectroscopy device 100, such as an illumination unit 102, a detection unit 108, a processing unit 109, or any other unit of the near-infrared spectroscopy device 100.

The computer system 200 may be a local computing device (e.g., a personal computer, laptop, tablet, or cell phone) with one or more processors and one or more storage devices, or may be a distributed computing system (e.g., a cloud computing system with one or more processors and one or more storage devices distributed at different locations, such as on a local client and/or one or more remote clients, as already mentioned). Certain embodiments may particularly include a distributed system including a near-infrared spectroscopy device 100, a smartphone, and a central or distributed server.

The computer system 200 may include any circuit or combination of circuits. In some embodiments, the computer system 200 may include one or more processors, which may be of any type, and may be integrated, as shown for processor 205, in a housing of the computer system or the near-infrared spectroscopy device 100. The term “processor” may, as used herein, refer to any type of computer circuit, such as a microprocessor, microcontroller, Complex or Reduced Instruction Set Microprocessor (CISC, RISC), Very Long Instruction Word (VLIW) or graphics processor, Digital Signal Processor (DSP), multicore processor, Field Programmable Gate Array (FPGA), for example in a near-infrared spectroscopy device 100 or a component thereof (e.g., an illumination unit 102 or a processing unit 109) or any other type of processing device. Other types of circuitries that may be included in the computer system 200 or any part of the near-infrared spectroscopy device 100, or of a further unit of a corresponding system 1, may be custom circuitry, an Application Specific Integrated Circuit (ASIC), or the like, such as one or more circuits (e.g., communication circuits) for use in wireless devices such as smartphones, tablet computers, laptops, two-way radios, and similar electronic systems.

The computer system 200, the near-infrared spectroscopy device 100, or any other unit forming part of system 1 may include one or more storage devices, such as illustrated for the storage device 206, which may include one or more memory elements suitable for a particular application, such as main memory in the form of random access memory (RAM), one or more hard disk drives, and/or one or more drives that operate with removable media, such as Compact Discs (CDs), flash memory cards, Digital Video Discs (DVD), and the like. The computer system 200, the near-infrared spectroscopy device 100, or any other unit forming part of system 1 may also include a display device, one or more speakers, a keyboard, and/or a controller, which may include a mouse, trackball, touchscreen, voice recognition device, or any other device that allows a user of the system to input information into and receive information, e.g., to and from the computer system 200.

According to some embodiments, methodologies for in-field determination of total sugars in green tobacco based on near-infrared spectroscopy are proposed. In certain embodiments, web, internet, or browser platforms as well as smartphone applications for the use of the portable near-infrared spectroscopy are provided, including a potential for new applications as discussed below.

According to some embodiments, the development of methods using near-infrared spectroscopy devices may in include three steps as illustrated in FIG. 2 in a flow diagram, which are in-field acquisition of sample spectra 21, reference analyses of these samples through conventional methods 22, and development of predictive models 23 using the spectra with their respective reference values.

According to some embodiments, a green tobacco dataset was set-up to include the influence of several factors (such as plant stage, leaf position, tobacco type, different devices, analysts, locations, and crops) to build robust and comprehensive prediction models. The main factors covered by the sampling are summarized in Table 1 and the number of samples of each type of tobacco are presented in Table 2.

TABLE 1
Factors included in the sampling for the database building.

| Crop | Tobacco Type | Leaf Position | Stage | Location |
|---|---|---|---|---|
| 2020 | Virginia | Bottom | Pre-flowering | Rio Negro |
| 2021 | Burley | Middle | After-topping | Mafra |
| | Comum | Top | | Nova Esperanca |
| | Amarelinho | | | Cruz Machado |
| | Dark | | | Sta. Cruz do Sul |
| | Maryland | | | Sta. Maria |
| | | | | Camaquã |

TABLE 2
Number of samples collected for the model calibration.

| Type | Region | Samples |
|---|---|---|
| Virginia | Camaqua/Sta. Cruz/Mafra/Rio Negro/Nova | 973 |
| | Esperança | |
| Amarelinho | Rio Negro | 108 |
| Burley | Sta. Maria/Cruz Machado/Rio Negro | 873 |
| Comum | Nova Esperança/Rio Negro | 273 |
| Dark | Mafra/Rio Negro | 224 |
| Maryland | Mafra | 107 |
| Total | | 2558 |

Tobacco types: VA—Virginia, AM—Amarelinho, BY—Burley, CO—Comum, DK—Dark, MD—Maryland.

As mentioned, in some embodiments a plurality of target spectra may be averaged for each of the tobacco target samples, which in an embodiment is three. In further embodiments, however, any other number of spectra may be averaged, but three has been proven to be a particularly suitable compromise between the target of smoothing out outliers and a larger number of spectra to be acquired. An average number of three may therefore result in a fast, yet reliable method. The best number of scans was decided based on the results of accuracy of the test set. Since there wasn't a significative difference between the accuracy using three or four scans, the model with three scans was chosen because the shorter analysis time. This is illustrated in table 3 below.

TABLE 3
Analysis time and accuracies for three and four samples averaged.

| No. of scans averaged | Analysis time | Accuracy in | Accuracy for |
|---|---|---|---|
| for each spectrum | (seconds) | cross-validation | test set |
| 3 | ca. 75 | 72% | 73% |
| 4 | ca. 100 | 75% | 77% |

Forming a first derivative in the context of preprocessing, in particular using a window size of fifteen and an order of two, has been proven to be of advantage in connection with preferred embodiments of the present invention as well. The best preprocessing was chosen based on the robustness of the model to spectral shifts and interferences. The model most robust to shifts plus interferences was developed using Savitzky-Golay (Savgol) first derivative with standard normal variate (SNV) and mean centering (MC), as illustrated in Table 4 below.

TABLE 4
Selection of preprocessing.

| | Robustness | Robustness to |
|---|---|---|
| Preprocessing | to shifts | interferences |
| Savgol (21^{a}, 2^{b}, 2^{c}) + SNV + MC | 11% | 50% |
| Savgol (15^{a}, 2^{b}, 1^{c}) + SNV + MC | 73% | 94% |
| Savgol (25^{a}, 2^{b}, 1^{c}) + MC | 100% | 36% |

^{a}window size;
^{b}polynomial order;
^{c}derivative order

In embodiments as disclosed herein, as mentioned, a wavelength range between 947 nm and 1,618 nm has been proved to be particularly advantageous. The near infrared device used according to embodiments of the present invention use covers 900 to 1700 nm, and removing the endmembers was found to be particularly advantageous to remove noise.

Be it noted that that selection of spectral variables is an approach that can be used to improve the performance of predictive models. Variable selection can be performed using simple rules, such as selecting the variables with the highest correlation with the property of interest, or through more sophisticated algorithms such as Interval Partial Least Square Regression (iPLS) or Genetic Algorithm (GA), among others. In the context of the present invention, variable selection by iPLS and GA was tested. However, this was found not to improve the model performance. Therefore, the model was kept with the range from 947 to 1,618 nm in embodiments of the present invention. Reference is made to table 5 below.

TABLE 5
Variable selection.

| Variable | Accuracy of | Robustness to | Robustness to |
|---|---|---|---|
| selection | test set | shifts | interferences |
| None | 73% | 48% | 71% |
| iPLS | 67% | 9% | 39% |
| GA | 73% | 5% | 16% |

Tobacco types were not, as may be done for analysing other compounds, grouped according to their curing process, such as flue curing and air curing, and were not analyzed separately for the determination of sugars. Particularly a separation into these different tobacco groups, i.e., training and using artificial intelligence models specifically selected for these tobacco groups, showed not to significantly improve correlation when analysing sugars.

In some embodiments, analyzing the tobacco target samples includes spectroscopically examining tobacco leaves at a plurality of predetermined sampling positions, which increased reproducibility and was found to be advantageous to reduce variation. Furthermore, according to some embodiments, scanning the tobacco target samples was performed under defined moisture conditions. In some embodiments, spectral acquisition is particularly performed applying a sampling head or window of a portable near-infrared device or similar spectroscopy device on green tobacco leaves folded in half at several different points, such as four points (from tip to stem), particularly on the lower leaf surface. The lower leaf surface may be advantageously analyzed due to the lower presence of surface moisture and absence of trichomes present on the upper surface which may comprise a different sugar content and reflectance.

Embodiments as disclosed herein are therefore advantageous to analyze target tobacco target samples which comprise one or more green tobacco leaves. These may particularly be scanned or probed under defined environmental conditions. Such environmental conditions may particularly include specific moisture conditions which may correspond to a period of the day used for the analysis. The scanning period was found to be advantageously to be performed between 9 a.m. and 5 p.m. to reduce the incidence of dew and higher moisture on the leaves, as surface moisture presents a significant signal in the near infrared region and results in interference in the analytical signal. However, other than a period during the day, other criteria or a direct measurement of the moisture may likewise be used.

As mentioned before, the method includes partial least squares regression which may be used as a basis of the one or more artificial intelligence models.

Embodiments of the method may particularly be performed in a system comprising a near-infrared spectroscopy device and a mobile unit connected or connectable to a server, wherein the trained artificial intelligence model may be downloaded from the server to the mobile unit to perform at least said deriving the concentration of the one or more sugars in the tobacco target samples.

In developing methods according to some embodiments, the leaf was removed from the plant, after taking the reference near-infrared spectra, stored in paper bags until sent for drying, peeling and grinding. Once the samples were prepared accordingly, a plurality of samples, which are 2,558 samples in a specific example, were analyzed by a reference method to obtain the results of total alkaloids and total sugars content. Then, 400 most representative samples were selected for determination of other analytes content (organic acids, polyphenols, aminoacids, carotenoids, and chlorophylls) by methods such as high-throughput simultaneous quantitation of multi-analytes in tobacco by flow injection coupled to high-resolution mass spectrometry (HTS-FIA-HRMS) as e.g. described in an article by Kaiser et al., Talanta 190, 2018, 363-374 (DOI: 10.1016/j.talanta.2018.08.007).

In some embodiments, prediction models were developed from the Partial Least Square regression (PLS) method. Different preprocessing (normalization, smoothing and derivatives), number of scans per sample, segregation by type of tobacco and selection of spectral variables were evaluated to obtain the models with the best predictive capacity, which were already discussed before. The performance of the models was evaluated using figures of merit related to accuracy, sensitivity and robustness obtained by the calibration set, cross-validation and external validation (33% of sample set). Table 6 illustrates the criteria or conditions considered in this connection.

TABLE 6
Conditions considered

| | | Tobacco | Variable |
|---|---|---|---|
| Preprocessing | Number of scans | type | selection |
| Smoothing | All scans | All | None |
| Smoothing, SNV* | Only 1^{st}/2^{nd}/3^{rd}/4^{th} | Bright | biPLS |
| 1^{st} derivative | Mean of 1^{st} and 2^{nd} | Earthy | GA |
| 1^{st} derivative, SNV | Mean of 2^{nd} and 3^{rd} | | |
| 2^{nd} derivative | Mean of 1^{st}, 2^{nd} and 3^{rd} | | |
| 2^{nd} derivative, SNV | Mean of 1^{st}, 2^{nd}, 3^{rd} and 4^{th} | | |

(SNV = Standard Normal Variates transformation)

Overall, 90 conditions per target were considered. The validation set was determined using the Onion algorithm. Best conditions were considered in view of accuracy and robustness for spectral shifts and interferences. The latter was determined using the inverse of the difference between maximum and minimum values Root Mean Square Error of Prediction (RMSEP). This value is a measure of the average uncertainty that can be expected when predicting new samples.

Advantageous conditions for the models were established through the accuracy, robustness and analytical frequency (i.e. lower number of scans per sample without loss of performance) values, which were already discussed before.

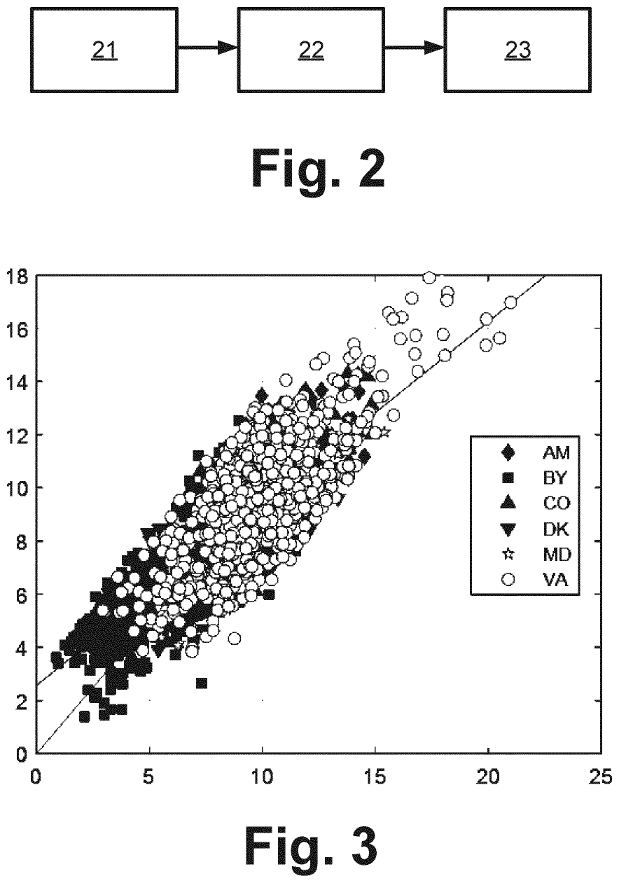

The figures of merit of the final models are presented in Table 4 and the fit plots between measured values (horizontal axis) and predicted values (vertical axis) are shown in FIG. 3.

In FIG. 3, diamonds indicate Amarelinho tobacco (AM), circles indicate Virginia tobacco (VA), squares indicate Burley tobacco (BY), triangles pointing upwards indicate Comum tobacco (CO), triangles pointing downwards indicate Dark tobacco (DK) and stars indicate Maryland (MD) tobacco. A fit is indicated with a straight line. The model presented a correlation coefficient of 82%. Therefore, the model presented good accuracy for the prediction of sugars in green tobacco leaves in the field. In other words, after the best conditions of preprocessing, number of scans, and variable selection were determined, the models were refined excluding outlier samples and optimizing model hyperparameters. Thus, a higher accuracy in the final model was achieved, as presented in Table 7.

TABLE 7
Parameters and figures of merit of total sugars.

| Curing | All |
|---|---|
| Scans | Mean of 3 scans |
| Spectral range | 947-1,618 nm |
| Min reference (% w/w) | 0.87 |
| Max reference (% w/w) | 21.0 |
| Correlation | 0.82 |
| Absolute error (% w/w) | 1.70 |
| Relative Error | 8% |

The models were also validated by different external sample sets in order to assess the robustness against NIR devices, analysts and crop not included in the models. The results of the external validation considering different situations are shown in Table 3. The prediction of total sugars for Virginia (VA) samples resulted in errors significantly equivalent to the absolute error of the model. Although the sugar prediction for Burley samples generated greater errors than for Virginia samples, the relative error was up to 15% (samples from Santa Maria). According to the external validation, the models for total sugars were robust against different crops, NIR devices and analysts, and presented sufficient accuracy for in-field analysis of new samples using NIR portable devices.

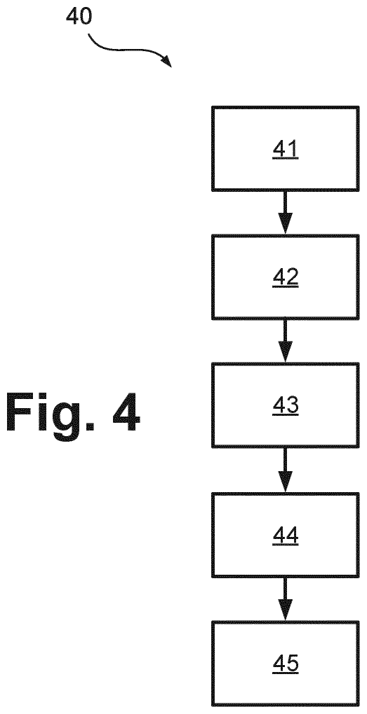

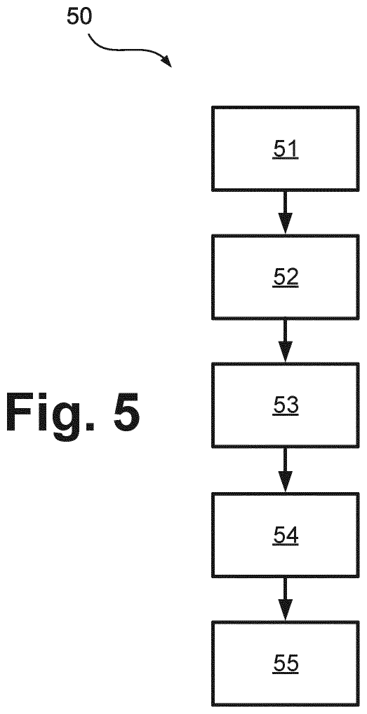

Summarizing what was said above, and with reference to FIG. 4 in which a corresponding method is indicated with reference numeral 40, according to embodiments of the present invention such a computer-implemented method includes a step 41 of providing one or more trained artificial intelligence models as described before and further illustrated in connection with FIG. 5, a step 42 of analyzing tobacco target samples using a target analysis technique providing near-infrared target spectra and a step 43 of preprocessing the near-infrared target spectra to obtain preprocessed near-infrared target spectral data, and a step 44 of deriving the concentration of the one or more sugars in the tobacco target samples using the or one of the trained artificial intelligence models and on the basis of the preprocessed near-infrared target spectral data. As to further details, reference is made to the explanations above.

As already mentioned, a method of providing one or more trained artificial intelligence models according to some embodiments is illustrated in FIG. 5 and referred to with 50. Method 50 may be used as method step 41, or a part thereof, as illustrated in FIG. 4. The method comprises a step 51 of providing a plurality of tobacco reference samples and sample information indicating characteristics of the tobacco reference samples, a step 52 of analyzing the tobacco reference samples for the one or more sugars using one or more reference analysis techniques providing reference concentration data, a step 53 of analyzing each of the tobacco reference samples using the target analysis technique providing near-infrared reference spectra, a step 54 of preprocessing the near-infrared reference spectra for the tobacco reference samples obtaining preprocessed spectral data and a step 55 of training one or more artificial intelligence models using the sample information, the reference concentration data and the preprocessed spectral data obtaining the trained artificial intelligence model. Again, as to further details, reference is made to the explanations above.

Some or all the steps of methods according to some embodiments may be performed (or used) by a hardware device such as, for example, a processor, a microprocessor, a programmable computer, or an electronic circuit. In some embodiments, some of the more steps of the method may be performed by such a device.

Depending on certain implementation requirements, some embodiments may be implemented by either hardware or software or both. The implementation may be carried out using a non-transitory storage medium, such as a digital storage medium such as a floppy disk, DVD, Blu-Ray, CD, ROM, PROM, EPROM, EEPROM, or FLASH memory, with electronically readable control signals recorded thereon that interact (or are capable of interacting) with a programmable computer system in a manner that executes an appropriate method. In this manner, the digital medium can be computer readable.

Some embodiments include a data carrier having electronically readable control signals that can interact with a programmable computer system such that one of the processes described herein is executed. In general, some embodiments may be implemented as a computer program product with program code, the program code being operative to perform one of the processes when the computer program product is executed on the computer. The program code may, for example, be stored on a machine-readable medium. Other embodiments include a computer program for performing one of the processes described herein stored on a machine-readable medium.

In other words, some embodiments relate to a computer program having program code for performing one of the methods described herein when the computer program is executed on a computer. Some embodiments may also relate a storage medium (or data medium or computer readable medium) having stored a computer program for performing one of the methods according to some embodiments when executed by a processor. The data carrier, digital medium or recorded medium may, in some embodiments, generally be tangible and/or non-transitory. Some other embodiments relate to a device as described herein comprising a processor and a storage medium.

Some other embodiments relate to a data stream or sequence of signals representing a computer program for performing one of the processes described herein. The data stream or signal sequence may, for example, be configured to be transmitted via a data connection, for example over the Internet. Yet another embodiment includes a processing means, such as a computer or programmable logic device configured or adapted to perform any of the processes described herein. Yet another embodiment includes a computer on which a computer program is installed to perform any of the processes described herein.

Yet other embodiments include a device or system configured to transmit (e.g., electronically, or optically) a computer program for performing one of the processes described herein to a receiver. The receiver may be, for example, a computer, a mobile device, a memory device, or the like. The device or system may, for example, include a file server to transmit the computer program to the receiver. In some embodiments, a programmable logic device (e.g., a field programmable gate array) may be used to perform all or part of the functionality of the methods described herein. In some embodiments, the programmable gate array may communicate with a microprocessor to perform any of the methods described herein. In general, the methods are preferably executed by any hardware device.

Implementation options and some embodiments can be based on the use of a machine learning model or a machine learning algorithm. The terms “artificial intelligence model” is used largely synonymously herein for some embodiments. Machine learning can refer to algorithms and statistical models that computer systems such as computer system 200 or any other suitable component forming part of a system 1 can use to perform a given task without explicit instructions, relying instead on models and inference.

For example, machine learning may use a data transformation derived from analysis of historical and/or training data instead of a rule-based data transformation. For example, near-infrared spectra may be analyzed using a machine learning model or algorithm. For a machine learning model to analyze near-infrared spectra, the machine learning model may be trained with near-infrared training spectra as input data and training content information as output data. By training the machine learning model with many near-infrared training spectra and/or features of near-infrared training spectra and suitable training content information (e.g., labels, annotations, sample information, contents information, substance information, quality information, quantity information), the machine learning model “learns,” in some embodiments, to associate features of near-infrared spectra, or the overall appearance of a spectrum, to sample features such as components contained in the sample and their contents, so that spectra not included in the training data can be recognized by the machine learning model. Particularly, near-infrared spectra may be provided in some embodiments, in connection with training a machine learning model, as image data, or in any other form conceivable and processable by machine-learning model.

Machine learning models can be trained using training data, as mentioned. Some embodiments may use a training method called supervised learning. In supervised learning, a machine learning model is trained using a set of training patterns, where each pattern may contain a set of input data values and a set of desired output values, i.e., each training pattern is associated with a desired output value. By specifying training patterns and desired output values, the machine learning model “learns” which output value to provide based on an input pattern that is similar to the patterns provided during training.

In addition to, or alternatively to, supervised learning, semi-supervised learning can also be used in some embodiments. In semi-supervised learning, some training patterns do not have a corresponding desired output value. Supervised learning can be based on a supervised learning algorithm (e.g., a classification algorithm, a regression algorithm, or a similarity learning algorithm). Classification algorithms may be used when the output values are restricted to a limited set of values (categorical variables), i.e., the input data is assigned to one of a limited set of values. Regression algorithms can be used when the outputs can have any numerical value (within a range). Similarity learning algorithms, which can be used in some embodiments, can resemble classification and regression algorithms, but are based on learning by example using a similarity function that measures how similar or related two objects are. In addition to, or alternatively to, supervised or semi-supervised learning, unsupervised learning can also be used in some embodiments to train a machine learning model. In unsupervised learning, (only) input data can be provided, and the unsupervised learning algorithm can be used to find structure in the input data (e.g., by grouping or clustering the input data, searching for commonalities in the data). Clustering is the division of input data consisting of multiple input values into subsets (clusters) such that the input values within a cluster are similar according to one or more (given) similarity criteria, while they are different from the input values in other clusters.

Reinforcement learning is a third group of machine learning algorithms, which can be used in some embodiments. In other words, reinforcement learning can be used to train a machine learning model. In reinforcement learning, one or more so-called software agents are trained to perform actions in an environment. Rewards are calculated based on the actions performed. In reinforcement learning, one or more software agents are trained to select actions that increase their overall rewards, making the software agents better at the task at hand (resulting in higher rewards).

In addition, some techniques can be applied to some machine learning algorithms according to some embodiments. For example, feature learning can be applied. In other words, the machine learning model can be trained at least partially by feature learning and/or the machine learning algorithm can include a feature learning component. The feature learning algorithms, which may also be referred to as view learning algorithms, store the information in the input but may also transform it to make it useful, often as a preprocessing step before performing classification or prediction. Feature training, for example, may be based on principal component analysis or cluster analysis.

In some examples, anomaly detection (i.e., outlier detection) may be used to identify suspect input values that deviate significantly from most input or training data. In other words, the machine learning model may be trained at least in part using anomaly detection and/or the machine learning algorithm may include an anomaly detection component.

In some examples, the machine learning algorithm may use a decision tree as a predictive model. In other words, the machine learning model may be based on a decision tree. In a decision tree, observations about an element (e.g., a set of input values) can be represented by branches of the decision tree, and the output value corresponding to the element can be represented by the leaves of the decision tree. Decision trees can support both discrete and continuous values as output values. When discrete values are used, the decision tree can be called a classification tree; when continuous values are used, the decision tree can be called a regression tree.

Associative rules are another technique that can be used in machine learning algorithms. In other words, a machine learning model can be based on one or more association rules. Association rules are created by finding relationships between variables in large data sets. A machine learning algorithm may define and/or use one or more relational rules that represent knowledge extracted from the data. For example, rules may be used to store, manipulate, or apply knowledge.

Machine learning algorithms are typically based on a machine learning model. In other words, the term “machine learning algorithm” can refer to a set of instructions that can be used to create, teach, or use a machine learning model. The term “machine learning model” may refer to a data structure and/or set of rules that represent learned knowledge (e.g., based on learning performed by a machine learning algorithm). In some embodiments of the invention, the use of a machine learning algorithm may include the use of an underlying machine learning model (or multiple underlying machine learning models). The use of a machine learning model may mean that the machine learning model and/or the data structure/set of rules representing the machine learning model have been trained by the machine learning algorithm.

For example, a machine learning model could be an Artificial Neural Network (ANN). ANNS are systems that mimic biological neural networks, such as those found in the retina or brain. Artificial neural networks may comprise a large number of interconnected nodes and a large number of connections, called edges, between the nodes. In general, there are three types of nodes, (1) input nodes that receive input values; (2) hidden nodes that are connected (only) to other nodes, and (3) output nodes that produce output values. Each node can represent an artificial neuron. Each edge can transmit information from one node to another. The output of a node can be defined as a (nonlinear) function of its inputs (e.g., the sum of its inputs). The inputs of a node can be used in a function based on the “weight” of the edge or node providing the input. The weights of nodes and/or edges can be adjusted during training. In other words, training an artificial neural network may particularly, and in certain embodiments, involve adjusting the weights of the nodes and/or edges of the artificial neural network, i.e., achieving the desired output for a given input.

Additionally, or alternatively, a machine learning model may be or include a support vector machine, a random forest model, or a gradient boosting model in some embodiments. Support vector machines (i.e., support vector networks) are supervised learning models with appropriate learning algorithms that can be used for data analysis (e.g., classification or regression analysis). Reference vector machines can be trained by providing a set of training input values that belong to one of two categories in some embodiments. A support vector machine can be trained to assign a new input value to one of the two categories. Additionally, or alternatively, in some embodiments the machine learning model may represent or include a Bayesian network, i.e., a probabilistic, directed, acyclic graph model.

A Bayesian network can represent a set of random variables and their conditional dependencies as a directed acyclic graph. Additionally, or alternatively, in some embodiments the machine learning model may be based on a genetic algorithm, a search algorithm, and a heuristic that mimics the process of natural selection.

Obtaining chemical information on green tobacco may conventionally require several steps, such as collection of representative leaves, refrigerated storage, transportation, drying, stalk removal, milling and analysis by laboratory reference methods, usually based on chemical reactions followed by visible-ultraviolet (UV-Vis) spectroscopy as well as infrared or chromatographic analyses. In addition to the long time, handling, and cost involved in conventional methods, the acquisition of chemical information on samples in places far from the laboratories is very difficult. Thus, the use of portable devices, which is envisaged according to certain embodiments, brings the benefit of analysis in remote locations, more representative sampling, and real-time information acquisition.

The various embodiments described herein are presented only to assist in understanding and teaching the claimed features. These embodiments are provided as a representative sample of embodiments only and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised, and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A computer-implemented method for detecting a concentration of one or more sugars in tobacco target samples using a target analysis technique based on near-infrared spectroscopy, the method comprising:
providing one or more trained artificial intelligence models obtained or obtainable by training one or more untrained or pre-trained artificial intelligence models using sample information, reference concentration data relating to the one or more sugars, and near-infrared reference spectral data obtained or obtainable using the target analysis technique for a plurality of tobacco reference samples;
analyzing the tobacco target samples using the target analysis technique providing near-infrared target spectra and preprocessing the near-infrared target spectra to obtain preprocessed near-infrared target spectral data; and
deriving the concentration of the one or more sugars in the tobacco target samples using the or one of the trained artificial intelligence models and on the basis of the preprocessed near-infrared target spectral data;
wherein said analyzing the tobacco target samples includes providing a plurality of the near-infrared target spectra in a common sub-range of the near-infrared wavelength range for each of the tobacco reference samples, and
wherein said preprocessing the near-infrared target spectra includes averaging said plurality of near-infrared target spectra for each of the tobacco target samples and performing a standard normal variates transformation.

2. The method according to claim 1, wherein the one or more sugars are selected from total sugars present in the tobacco target samples and glucose.

3. The method according to claim 1, wherein the plurality of near-infrared target spectra averaged for each of the tobacco target samples is three.

4. The method according to claim 1, wherein said preprocessing further comprises a standard normal variate transformation, followed by Savitzky-Golay first derivative and mean centering.

5. The method according to claim 4, wherein a window size of fifteen and an order of two is used in forming the first derivative.

6. The method according to claim 1, wherein the common sub-range of the near-infrared wavelength range is between 947 nm and 1,618 nm.

7. The method according to claim 1, wherein said groups include at least one of a flue curing and an air curing group.

8. The method according to claim 1, wherein said analyzing the tobacco target samples includes spectroscopically examining tobacco leaves at a plurality of predetermined sampling positions.

9. The method according to claim 1, wherein said analyzing the tobacco target samples is performed under defined moisture conditions.

10. The method according to claim 1, wherein the tobacco target samples comprise one or more green tobacco leaves.

11. The method according to claim 10, wherein the one or more green tobacco leaves are probed under defined probing conditions.

12. The method according to claim 1, wherein the method includes partial least squares regression.

13. The method according to claim 1, wherein the method is performed in a system comprising a near-infrared spectroscopy device and a mobile unit connected or connectable to a server, wherein the one or more trained artificial intelligence models is downloaded from the server to the mobile unit to perform at least said deriving the concentration of the one or more sugars in the tobacco target samples.

14. A method of providing one or more trained artificial intelligence models usable in the method according to claim 1, the method comprising:
providing a plurality of tobacco reference samples and sample information indicating characteristics of the tobacco reference samples;
analyzing the tobacco reference samples for the one or more sugars using one or more reference analysis techniques providing reference concentration data;
analyzing each of the tobacco reference samples using the target analysis technique providing near-infrared reference spectra;
preprocessing the near-infrared reference spectra for the tobacco reference samples obtaining preprocessed spectral data; and
training one or more artificial intelligence models using the sample information, the reference concentration data and the preprocessed spectral data obtaining the trained artificial intelligence model;
wherein said analyzing the tobacco reference samples and said preprocessing the near-infrared reference spectra is performed as for the tobacco target samples.

15. A trained artificial intelligence model stored on a non-tangible computer-readable medium obtained or obtainable by using the method according to claim 14.

16. The method according to claim 1, wherein the one or more trained artificial intelligence models is applied to a new sample by multiplying a vector of regression coefficients and a pre-processed average spectrum of the sample, generating a target concentration as a result.

17. A system for detecting a concentration of one or more sugars in tobacco target samples using a target analysis technique based on near-infrared spectroscopy, the system being configured to perform the method according to claim 1.

18. The system according to claim 17, comprising a near-infrared spectroscopy device and a mobile unit connected or connectable to a server, wherein the one or more trained artificial intelligence models is downloadable from the server to the mobile unit and wherein the mobile unit is configured to perform at least said deriving the concentration of the one or more sugars in the tobacco target samples.

19. A computer program with a program code for performing the method according to claim 1 when the computer program is run on a processor.
